(19) United States
(12) Patent Application Publication
(10) Pub. No.: US 2003/0032662 A1
(43) Pub. Date: Feb. 13, 2003

(54) Micronized leflunomide

(76) Inventor: Ilya Avrutov, Bat Hefer (IL)

Correspondence Address: KENYON & KENYON, ONE BROADWAY, NEW YORK, NY, 10004, US

(21) Appl. No.: 10/269,696

(22) Filed: Oct. 10, 2002

Related U.S. Application Data
(63) Continuation of application No. 09/929,409, filed on Aug. 14, 2001, now abandoned.
(60) Provisional application No. 60/225,372, filed on Aug. 14, 2000.

(51) Int. Cl. A61K 31/42

(52) U.S. Cl. 514/378

(57) ABSTRACT

The present invention relates to leflunomide having relatively small particles, and corresponding relatively large surface area. In one embodiment, the invention relates to leflunomide and formulations containing leflunomide having a mean particle diameter of less than 200 micrometers.

### CROSS-REFERENCE TO RELATED APPLICATION

The present application claims benefit of U.S. provisional application Ser. No. 60/225,372, filed Aug. 14, 2000, which is incorporated by reference.

### FIELD OF THE INVENTION

This invention relates to micronized leflunomide and to the preparation thereof.

### BACKGROUND OF THE INVENTION

Leflunomide, N-(4′-trifluoromethylphenyl)-5-methylisoxazole-4-carboxamide, having the formula 1:

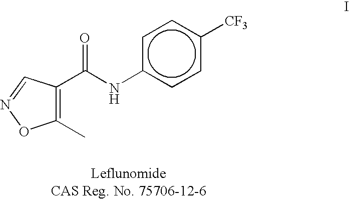

is approved, under the trademark ARAVA®, by the U.S. Food and Drug Administration, for the treatment of rheumatoid arthritis. Leflunomide is a pyrimidine synthase inhibitor with antiproliferative activity. Leflunomide metabolizes to 2-cyano-3-hydroxy-N-[4-(trifluoromethyl)phenyl)]-(2E)-butenamide in the human body. The metabolite 2-cyano-3-hydroxy-N-[4-(trifluoromethyl)phenyl)]-(2E)-butenamide is the active agent that inhibits pyrimidine synthase.

U.S. Pat. No. 4,284,786 discloses a process for making leflunomide, the contents of which are incorporated by reference. The reference, Pharmaceutical Dosage Forms: Tablets, Vol. 2, 2^{nd} Ed., Lieberman et al. Ed., Marcel Dekker, Inc, New York, (1990) p. 107-200; and Australian patent No. AU-A-78870/98, are incorporated herein by reference.

The Particle Size Distribution (PSD) of leflunomide crystals may be used to determine the available surface area for the drug dissolution. Often, it is observed that the available surface area for drug dissolution correlates to the rate of dissolution and solubility where a greater surface area enhances the solubility of a drug and enhances the rate of dissolution of a drug. Further, the velocity of dissolution of a drug often effects the drug's bioavailability. Thus, the PSD of leflunomide, and in particular, the mean particle diameter, are important parameters to characterize and predict the bioavailability of the drug. It is desirable to have leflunomide with a particle size in which the mean particle size enhances the reproducibility of; (a) the rate of dissolution and (b) the reproducibility of the dissolution. It is desirable to have leflunomide in which the mean particle size imparts an improved and stable dissolution profile.

### SUMMARY OF THE INVENTION

An object of the present invention is to provide leflunomide formulations containing leflunomide having relatively small particles, and corresponding relatively large surface area.

It is also an object of the present invention is to provide leflunomide with a particle size in which the mean particle size enhances the reproducibility of, (a) the rate of dissolution and (b) the reproducibility of the dissolution.

It is also an object of the present invention to provide leflunomide in which the mean particle size imparts an improved and stable dissolution profile.

The present invention provides leflunomide and formulations containing leflunomide having a mean particle diameter of less than 200 micrometer.

The present invention also provides leflunomide and formulations containing leflunomide having a mean particle diameter of less than 20 micrometers.

The present invention also provides processes for preparing micronized leflunomide.

The present invention also provides processes for preparing micronized leflunomide, where in the leflunomide to be micronized is pure leflunomide.

The present invention also provides processes for preparing micronized leflunomide, where in the leflunomide to be micronized is dry leflunomide.

The present invention also provides pharmaceutical compositions comprising micronized leflunomide.

### DETAILED DESCRIPTION OF THE INVENTION

The present invention provides leflunomide formulations containing leflunomide having relatively small particles, and corresponding relatively large surface area.

The present invention also provides leflunomide and formulations containing leflunomide having a mean particle diameter of less than 200 micrometer, preferably the mean particle diameter is less than 100 microns, more preferably the mean particle diameter is less than 20 microns, most preferably the mean particle size is about 10 microns.

The present invention also provides leflunomide having a mean particle diameter of between about 200 microns and about 10 microns. In another embodiment of the present invention, leflunomide has a mean length of about 4.2 microns, more preferably a mean length of 4.0 microns. The term “micronized” is used herein as referring to particles having a mean particle diameter of less than about 200 microns. Micronized particles of leflunomide, may be obtained by methods disclosed in U.S. Pat. No. 5,834,472, the contents of which are incorporated herein by reference.

The present invention also provides processes for preparing micronized leflunomide. By the methods of the present invention, leflunomide, which is prepared by methods known in the art, is separated by sieves to produce leflunomide wherein 50% has a mean particle diameter of below about 250 microns and about 80% has is below about 500 microns. The sieved leflunomide is then micronized by methods known in the art, e.g., in a micronizer, to yield leflunomide wherein 100% of the leflunomide has a mean particle size of less than about 45 microns, preferably 99% of the leflunomide has a mean particle size of less than about 45 microns, more preferably, 93% of the leflunomide has a mean particle size of less than about 7.5 microns, more preferably the leflunomide isolated has a mean particle diameter of less than 10 micron.

Micronized particles of leflunomide can be obtained by the use of conventional micronizing techniques after sieving to provide leflunomide wherein about 50% has a particle size less than 250 microns and about 80% has a particle sized below about 500 microns. By the methods of the present invention, the leflunomide where about 50% has a particle size less than 250 microns and about 80% has a particle sized below about 500 microns, is micronized to the desired particle size range by methods known in the art, for example, using a ball mill, ultrasonic means, fluid energy attrition mills, or using a jet mill, or other suitable means as disclosed in Pharmaceutical Dosage Forms: Tablets, Vol. 2, 2^{nd} Ed., Lieberman et al. Ed., Marcel Dekker, Inc, New York, (1990) p. 107-200, the contents of which are incorporated herein by reference.

The present invention also provides processes for preparing micronized leflunomide, wherein the micronized leflunomide is made from dry leflunomide.

In accordance with the present invention, the present micronized leflunomide may be prepared as pharmaceutical compositions that are particularly useful for the treatment of hypertension and edema associated with congestive heart failure, renal disease, or hepatic disease. Such compositions comprise micronized leflunomide with pharmaceutically acceptable carriers and/or excipients known to one of skill in the art.

In accordance with the present invention, the present micronized leflunomide may be prepared as pharmaceutical compositions that are particularly useful for the treatment of active rheumatoid arthritis (RA). Such compositions comprise micronized leflunomide with pharmaceutically acceptable carriers and/or excipients known to one of skill in the art.

Preferably, these compositions are prepared as medicaments to be administered orally, or intravenously. Suitable forms for oral administration include tablets, compressed or coated pills, dragees, sachets, hard or gelatin capsules, sub-lingual tablets, syrups and suspensions. While one of ordinary skill in the art will understand that dosages will vary according to the indication, age of the patient, etc., generally micronized leflunomide of the present invention will be administered at a daily dosage of about 10 to about 25 mg per day, and preferably about 20 to about 25 mg per day.

### EXAMPLES

The present invention will now be further explained in the following example. However, the present invention should not be construed as limited thereby. One of ordinary skill in the art will understand how to vary the exemplified preparations to obtain the desired results.

### Example 1

Pure dry leflunomide was micronized in a micronizer. The result was micronized leflunomide of a particle size of less than 5 micrometer.

It should be understood that some modification, alteration and substitution is anticipated and expected from those skilled in the art without departing from the teachings of the invention. Accordingly, it is appropriate that the following claims be construed broadly and in a manner consistent with the scope and spirit of the invention.

What is claimed is:

1. Micronized leflunomide.
